(19) United States
(12) Patent Application Publication
(10) Pub. No.: US 2026/0201304 A1
(43) Pub. Date: Jul. 16, 2026

(54) A ROTATING TISSUE CULTURE INSERT FOR THE POSITION INSIDE A ROTATABLE ROLLER BOTTLE PARTIALLY FILLED WITH A LIQUID FOR THE CULTIVATION OF CELLS

(71) Applicant: UNIVERSITÄT ZÜRICH, Zürich (CH)

(72) Inventors: Emanuela S. FIORETTA, Schlieren (CH); Nikolaos POULIS, Schlieren (CH); Maximilian Y. EMMERT, Schlieren (CH); Simon P. HOERSTRUP, Schlieren (CH)

(21) Appl. No.: 19/133,081

(22) PCT Filed: Oct. 18, 2023
(86) PCT No.: PCT/EP2023/079051

§ 371 Date: May 27, 2025

(30) Foreign Application Priority Data
Nov. 28, 2022 (EP) 22209906.1

(51) Int. Cl. C12M 3/04; C12M 1/12; C12M 3/00

(52) U.S. Cl. CPC C12M 27/12; C12M 21/08; C12M 25/14

(57) ABSTRACT

A rotating tissue culture insert for positioning inside a rotatable roller bottle partially filled with a liquid for the cultivation of cells includes a scaffold provided for the cultivation of cells, an axle body, and at least two protrusions extending in radial direction from the axle body. The protrusions define wheels of the rotating tissue culture insert with a runway that is in contact with the surface of a roller bottle when the roller bottle is in a lying position.

### TECHNICAL FIELD

Exemplary embodiments of the invention relate to a rotating tissue culture insert for the position inside a rotatable roller bottle partially filled with a liquid for the cultivation of cells.

Roller bottles are commonly used for about a decade or even longer as method and in a machine for the cultivation of cells. Usually, roller bottles are used for cell growth. In some rare applications the roller bottles may comprise a liquid for the cultivation of cells with nutrients and/or cells and a scaffold for the deposition of the cells. The roller bottle is usually used for the cultivation of cells in a lying position. That means that the roller bottle is rotated around a horizontal rotation axis that is parallel to the longitudinal axis of the bottle, which is commonly cylindrical.

After the growth of a tissue at the surface of the scaffold, cells are removed in a subsequent step by decellularization. The tissue, without living cells can be used in various medical applications, such as for implantation or as a dressing or the like, without immunicompatibility issues. In some applications there is a demand for a modelized tissue for the replacement of organs. In some other applications there is also a demand of tissue with a specific collagen orientation.

The inventive rotating tissue culture insert can be for the fabrication of tissue for all the aforementioned applications.

### BACKGROUND OF THE INVENTION

Roller bottles and an apparatus for the movement of roller bottles are well known in the art. The state-of-the art technology in this field has focused thus far on adaptation of the roller bottle itself, as disclosed in EP 0 394 713A1 . This document discloses a porous polystyrene foam being the scaffold.

Further methods for engineering tissues are disclosed by AU 2013277275 B2 and by AU 2017202721A1 .

A further bioreactor for the production of is disclosed by U.S. Pat. No. 8,192,981 B2 for the manufacturing of (stented) TEHVs.

### SUMMARY OF THE INVENTION

It is one first object of the current invention to provide the possibility to impose a preferential ECM orientation in longitudinal direction.

It is one second object of the current invention to provide the possibility to impose a preferential ECM orientation in circumferential direction.

A third object of the current invention is to enhance medium perfusion and oxygenation.

It is a fourth object of the current invention to simplify a method of using a roller bottle for the cultivation of cell-based tissue constructs in comparison to today's standards in this field of technology. The tissues comprise all cell-based matrix constructs which can potentially be produced by the method described hereinafter.

An inventive rotating tissue culture insert, short RTCI, is adapted for the position inside a rotatable roller bottle that is partially filled with a liquid for the cultivation of cells. The bottle is not part of the inventive insert. The rotating insert is adaptable to rotate inside the roller bottle. The roller bottle is typically in a lying position, in other words the bottle has a horizontal orientation.

A tissue culture insert is provided with a surface, typically a scaffold, on which the cells can accumulate and/or grow. The insert comprises a scaffold provided for the cultivation of cells preferably at its surface but also into the scaffold material. The scaffold can preferably formed from biodegradable material that degrades during the formation and growth of the tissue.

The RTCI may further comprise a scaffold holder for holding the scaffold.

The scaffold holder can preferably be a mesh-like cylindrical support used to hold the scaffold. The scaffold holder can more preferably be a nitinol stent. The nitinol stent might be covered by a non-adhesive coating and/or layer to favor scaffold removal. The non-adhesive layer can be preferably made of parafilm.

The nitinol stent used as an integral part of the production process may in the end product serve as an anchoring system such as an implantation stent system for clinical use. The nitinol material is merely one preferred example for a stent.

According to the invention the rotating tissue culture insert comprises an axle body and at least two protrusions extending in radial direction from the axle body.

Further parts, such as a scaffold holder might also be part of said rotating tissue culture insert.

The protrusions form two wheels of the RTCI. The wheels are provided with a runway that is in contact with the surface of a roller bottle when the roller bottle is in an essentially lying resp. horizontal position.

The invention provides the ability to create a tissue engineered product with a desired preferential collagen orientation by using a RTCI in combination with a roller bottle system. It further allows the rotation of the scaffold with the roller bottle, to enhance medium perfusion and oxygenation and/or enables to impose a preferential ECM orientation via static stretch.

According to a preferred embodiment the RTCI can be used to provide a preferential longitudinal ECM orientation or in another preferred embodiment the RTCI may apply a static circumferential stretch over tissue culture time to induce a preferential circumferential ECM orientation.

A preferred application is the use of the insert for the manufacturing in the cardiovascular tissue engineering field, such as the production of a tissue engineered heart valve, short TEHV.

For the use of the maximal surface of the insert, it is of advantage that at least two protrusions are at a distal position of the axle body. A distal position is an end position of a body.

The maximal radial height of the protrusions is larger than thickness of the scaffold. In the case that an additional substrate also referred to as scaffold holder for holding the scaffold is used, which is positioned over the outer surface of the axle body, the radial dimensions of the protrusions is larger than the sum of the thickness of the scaffold and the substrate. A further optional part of the insert might be a sleeve such as a multipart sleeve, being positioned coaxially to the axle body. If the sleeve is part of the axle body, then the protrusions are larger than the sum of the scaffold, the substrate and the sleeve.

It is advantageous if the substrate and/or the sleeve comprises a plurality of openings. Meshes are also understood as openings in the context of the current inventions and are preferred as a material for the substrate holder. The substrate is also referred to as a scaffold holder, preferably a demountable part of the rotating tissue culture insert. The scaffold holder can be individually designed according to the form, size, and function of an implant to be formed by a combination of the tissue and the substrate. In this case, the substrate or in other words the scaffold holder can form an arrangement together with the tissue, which can be demounted from the rotating tissue culture insert after the formation of the tissue.

Alternatively, or additionally the sleeve, being preferably a multipart-sleeve is provided with a longitudinal axis parallel to the axle body and comprises a plurality of openings and wherein the scaffold holder is preferably positioned over the sleeve. If there is no sleeve the scaffold holder can be positioned over the axle body and is more preferably part of the rotating tissue culture insert

Alternatively, the formed tissue can be detached from the substrate and can be used for implantation or other medical use without the scaffold holder.

For optimal rotation properties the axle body is a hollow axle body. For an optimum contact of the tissue with the liquid inside the roller bottle the hollow axle body may be provided with a plurality of openings.

Since the scaffold and/or the substrate may have different thickness depending on the tissue to be formed, the protrusions are demountable from the axle body and preferably connected to the axle body by a thread. This way an axle body can be provided with protrusions of different dimensions. Thus, the rotating tissue culture insert can be designed in different dimension by using the same axle body.

Preferably the protrusions have the form of circumferential rings.

The scaffold may further comprise a nonwoven layer so that the liquid can easily penetrate the surface of the scaffold. Preferably the nonwoven can be made of biodegradable material. It may comprise or consist of polyglycolic acid.

In a further preferred embodiment the nonwoven layer can be provided with a coating, more preferable with a biodegradable coating, most preferable with a polyhydroxybutyrate-film.

Alternatively, or advantageously to the idea of the protrusions forming two wheels, the rotating tissue culture insert can be provided as a stretcher for stretching the scaffold in radial direction.

This way the system allows for the possibility to easily adjust the rotating tissue culture insert size in relation to the approach, i.e.: smaller or larger stent-like scaffold holders or in other words support structures can be used to achieve the desired outcome, without the need to change the roller bottle system. Moreover, disposable roller bottle flasks are easy to use, largely available, and can accommodate a great variety of scaffold sizes.

Preferred embodiments of the previously described invention and/or embodiments in advantageous combination with the idea of the protrusions in form of wheels, are described by the further dependent claims.

It is of advantage that axle body comprises a longitudinal axis and wherein the rotating tissue culture insert further comprises one or more parts that are radially movable in relation to the longitudinal axis.

In an advantageous constructional concept, the rotating tissue culture insert may comprise an adjustment mechanism for the adjustment of the radial distance of the one or more part in relation to the longitudinal axis of the axle body.

For a better distribution of the force applied to the scaffold, the one or more parts are arcuated plates.

For a better contact of the scaffold with the liquid the arcuated plates might be provided with openings.

In a preferred embodiment the axle body can be part of the adjustment mechanism and is formed by at least two elements that are linear movable to each other and wherein the elements preferably comprise conical segments at each the element. The adjustment mechanism can be adjustable by screwing or in other word by a screwing mechanism.

In a further embodiment of the invention or as an independent inventive idea the substrate can be essentially cylindrical and may comprise different sections of bendability, preferably bendability in radial direction, more preferable different sections of bendability in a distal position of the substrate.

This might be achieved for example by a mesh structure comprising different mesh sizes.

A further idea of the invention is the provision of a method for the fabrication of an implant wherein the method comprises the following steps:
A: Providing a roller bottle comprising a liquid for the cultivation of cells and a rotating tissue culture insert comprising a rotating tissue culture insert provided with a scaffold, wherein the roller bottle is in a lying position, such that the fill level of the roller bottle is essentially parallel to the longitudinal axis of the bottle. The RTCI may be the inventive RTCI as previously described.
   The rotating tissue culture insert further comprises a substrate also referred to as a scaffold holder for the attachment of the scaffold at the rotating tissue culture insert.
B: Rotation of the roller bottle, wherein the scaffold is distanced towards the wall of the bottle and wherein the insert is rotating, preferably counter-clockwise to the direction of rotation of the roller bottle, and wherein a tissue is formed at the surface of the scaffold during the rotation of the roller bottle;
C: Removal of an arrangement comprising the tissue and the substrate from the rotating tissue culture insert;
   According to the invention the arrangement is provided for the formation an implant. This means that the arrangement is already the implant or can be transformed to be an implant.

The aforementioned arrangement may comprise an essentially cylindrical section wherein radial diameter of the rotating tissue culture insert is adjustable and wherein the radial diameter of the rotating tissue culture insert is adjusted to a variable diameter of the cylindrical section.

### BRIEF DESCRIPTION OF THE DRAWINGS

Some advantageous embodiments for an inventive rotating tissue culture insert and an inventive embodiment of a method for fabrication are further explained in detail below together with drawings. Specific parts of the different embodiments can be understood as separate features that can also be realized in other embodiments of the invention. The combination of features described by the embodiment shall not be understood as a limitation for the invention.

FIG. 1 illustrates a scaffold according to embodiments of the invention;

FIG. 2 illustrates a hollow axle body according to embodiments of the invention;

FIG. 3 illustrates a substrate according to embodiments of the invention;

FIG. 4 illustrates a scaffold according to exemplary embodiments of the invention;

FIG. 5 illustrates a rotating tissue culture insert according to exemplary embodiments of the invention;

FIG. 6 illustrates the inner surface of a roller bottle according to exemplary embodiments of the invention;

FIG. 7 illustrates an axle body according to exemplary embodiments of the invention;

FIG. 8 illustrates a rotating tissue culture insert according to exemplary embodiments of the invention;

FIG. 9 illustrates arcuated plates according to exemplary embodiments of the invention;

FIG. 10 illustrates an axle body and arcuated plates according to exemplary embodiments of the invention;

FIG. 11 illustrates parts of a scaffold according to exemplary embodiments of the invention;

FIG. 12 illustrates a hollow axle body according to exemplary embodiments of the invention;

FIG. 13 illustrates a rotating tissue culture insert according to exemplary embodiments of the invention;

FIGS. 14a-14c illustrate tested samples after culturing for two weeks, four weeks, and 6 weeks, respectively, according to exemplary embodiments of the invention;

FIG. 15 illustrates graphs of results of testing according to exemplary embodiments of the invention;

FIGS. 16a-16c illustrate a histological evaluation of samples cultured for six weeks according to exemplary embodiments of the invention;

FIGS. 17a-17c illustrate a histological evaluation of the samples in FIGS. 16a-16c cut in the circumferential direction according to exemplary embodiments of the invention;

FIG. 18 illustrates a substrate according to exemplary embodiments of the invention;

FIG. 19 illustrates a substrate according to exemplary embodiments of the invention; and

FIG. 20 illustrates a valve flap according to exemplary embodiments of the invention.

### DETAILED DESCRIPTION

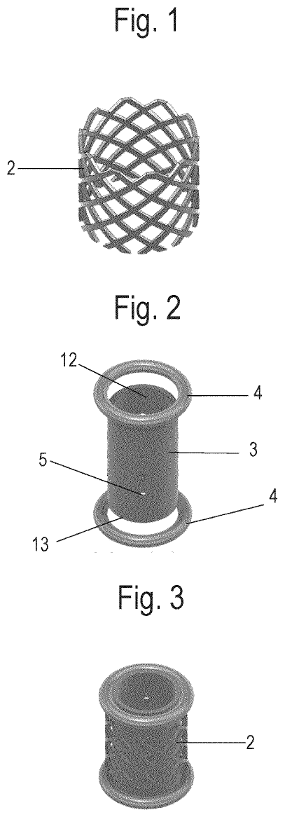

FIG. 1 discloses a first embodiment with a substrate 2 supporting a scaffold 11, preferably in the form of a stent. The substrate 2 of this embodiment has the preferred form of a cylindrical mesh body. The substrate 2 can be combined with further parts to a rotating tissue culture insert 20 as shown in FIG. 5.

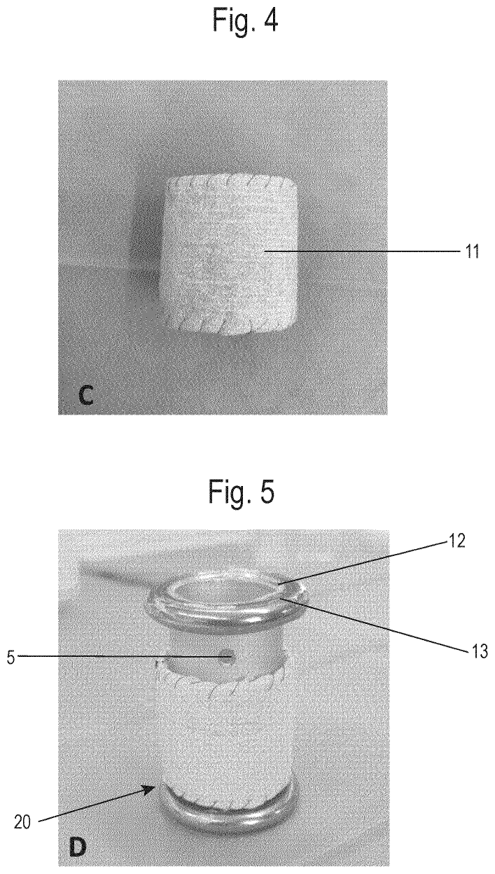

The scaffold 11 (FIG. 4) can be mounted to the substrate 2 of the specific form shown in FIG. 1 using a medical thread by sewing or the like, although other methods of fixation are also possible.

The scaffold rotating tissue culture insert 20 further comprises a hollow axle body 3 as shown in FIG. 2. The substrate 2 can be positioned over the outer surface of the hollow axle body 3.

The rotating tissue culture insert is provided with at least two rolling protrusions 4 which are positioned parallel to each other at the hollow axle body. These protrusions 4 are preferably arranged circumferentially around the hollow axle body 3, thus forming a ring shape. The protrusions 4 are firmly connected to the hollow axle body 3
Each of the protrusions 4 has at least a circumferential runway on which the respective rolling protrusions can be in contact and can roll in contact with a surface, such as the inner wall of a roller bottle. The runway can be interrupted by grooves. The protrusions 4 are provided to be wheels firmly connected to the axle body 3. The substrate 2 and the scaffold 11 can be placed between the protrusions, wherein the radial height of the protrusions is bigger than the sum of the thickness or-in other words—the radial height of the substrate and the scaffold combined.

At least two of the protrusions 4 can be mounted at each end of the hollow axle body 3. They are in a distal position. In a preferred embodiment the mounting the protrusions can be done either by transition or interference fit, by screwing and/or by a combination of clearance fit and welding.

Further protrusions, preferably having an identical form to the protrusions 4, can be mounted preferably with equidistant from each other and from the distal positioned protrusions 4.

As shown in FIG. 2 the hollow axle body 3 is provided with openings 5 in the shell surface. The openings are preferably arranged in a row running parallel to the longitudinal axis of the hollow axle body. One opening 5 of one row is equidistant to the neighboring openings of the row. There are a number of rows of openings circumferentially distributed over the shell surface of the hollow axle body 3.

The material of some parts, preferably all parts of the rotating tissue culture insert 20 might be a metal, preferably of stainless steel for medial use.

The scaffold 11 is shown in FIG. 4. It can be made of a polymeric material, preferably a nonwoven, more preferably comprising polyglycolic acid (PGA). The PGA-nonwoven might preferably be coated with a poly-4-hydroxybutyrate as described later on as a biodegradable film for the control of the cell matrix adhesion. The distal ends 12 is provided with threads 13. The scaffold 11 has the preferred form of a hollow cylinder but might have an alternative prismatic form.

FIG. 5 discloses a rotating tissue culture insert 20, briefly called RTCI, which comprises the scaffold 11. This insert can be used in a rotating cell culture system, RCCS, where a rotating bioreactor is used for the growth of the cells. The rotating bioreactor can be formed by a roller bottle, as shown for example in EP 0 394 713. The invention is not limited to the use of the RTCI in the roller bottles disclosed in this document.

Roller bottles are known in the art. These containers are used in the laboratory and like situations for culturing of cells. They are generally cylindrically shaped and are adapted to rotate about their axes. The internal surfaces of such roller bottles are for providing active surfaces for the growth of cells. A liquid growth medium is introduced into the roller bottles. The rotating movement of the bottle keeps the internal surfaces wetted with the liquid medium, thereby encouraging the growth of cells. Rotating rollers in an appropriate apparatus are employed to rotate these roller bottles. Usually, the roller bottle apparatus is adapted to be placed inside an incubator or incubating room to control the temperature of cell growth inside the roller bottles. A rotational driving system, such as rotating rollers, as a part of the apparatus is employed to rotate these roller bottles.

By turning the roller bottle with the RTCI, the RTCI will rotate counter-clockwise or clockwise to the direction of rotation of the roller bottle inside the roller bottle.

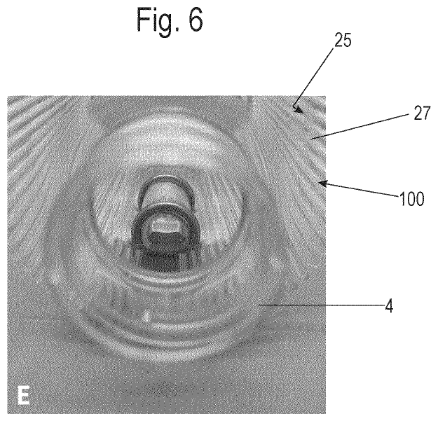

FIG. 6 discloses the inner surface 25 of such a roller bottle 100. The inner surface is provided with ribbons 27 extending parallel to the axis of rotation and the longitudinal axis of the roller bottle 100. The ribbons 27 are provided for stopping the rotation of the RTCI once the roller bottle is stopped. An axle body with protrusions 4 is here shown made of transparent plastic material to show the arrangement and function of the RTCI inside the roller bottle 100.

The roller bottle is preferably provided with a neck, such that the bottle is able to withhold of the liquid for cultivating cells in horizontal position. The RTCI is designed in a dimension such that the RTCI preferably rotates at least 2.5 times, preferably 3-4 times around its axis, while the bottle performs one rotation. The RTCI should be in a dimension that it passes the neck of the roller bottle.

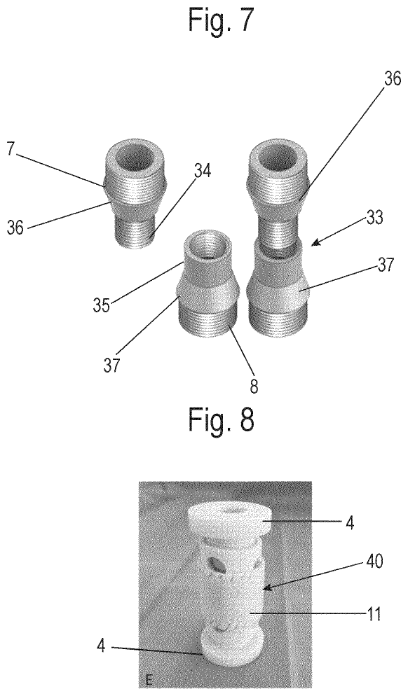

FIGS. 7-11 illustrate a second embodiment of the current invention, wherein a multipart sleeve 32 is a part of an made of a further embodiment of the rotating tissue culture insert 40 comprising arcuated plates 6 as a part of a jacket section. Each plate is provided with a number of openings 5, preferably at least a row of openings 5. A scaffold holder, or in other words a substrate, can be placed over the sleeve 32.

In the embodiment of FIG. 7-11 there are four arcuated plates 6, however there could be another number of plates thus forming the sleeve. The sleeve cooperates with an axle body 33, for the adjusting the diameter of the sleeve 32 and the gap between the plates 6. The axle body 33 is formed by an arrangement of at least two parts 7, 8 that are linear movable to each other. In the current invention the linear movability can be achieved by screwing the two parts 7, 8 together. Therefore, one part 7 is provided with a screw bolt 34 and the second part is provided with a screw sleeve 35. The previously described screwing connection of the parts can also be achieved by similar arrangements such as one central screw pin and two distal screw sleeves or by one central screw sleeve and two distal parts provided with screw bolts.

Two parts 7, 8 of the arrangement are provided with conical sections 36, 37 which axial distance can be varied, preferably by screwing or unscrewing the parts. When approaching the conical sections 36, 37 the arcuated plates 6 are spread or respectively are further spaced apart from each other and thus have a higher radial distance from each other compared to the case that the conical sections 36, 37 are further spaced apart from each other.

Because there is sufficient space between axle body 33 and the sleeve 32 formed by the actuated plates 6, the axle body 33 does not need to have a hollow shape or openings 28 of the rotating tissue culture insert 40 of FIG. 7-11 compared to the embodiment of a rotating tissue culture insert 20 of FIG. 1-6, although a hollow shape of the axle body 33 is also preferred in this embodiment.

At the distal positions of the axle body 33 is provided with a thread for mounting rolling protrusions 4 similar to FIG. 1-6. This way the protrusions can be demounted and cleaned independently for reuse. Especially the connection parts are difficult to clean in an acceptable way, therefore a demountability of all parts of the RTCI is preferred. The parts including the scaffold 11 are shown in FIG. 11 and mounted to an RTCI in FIG. 13.

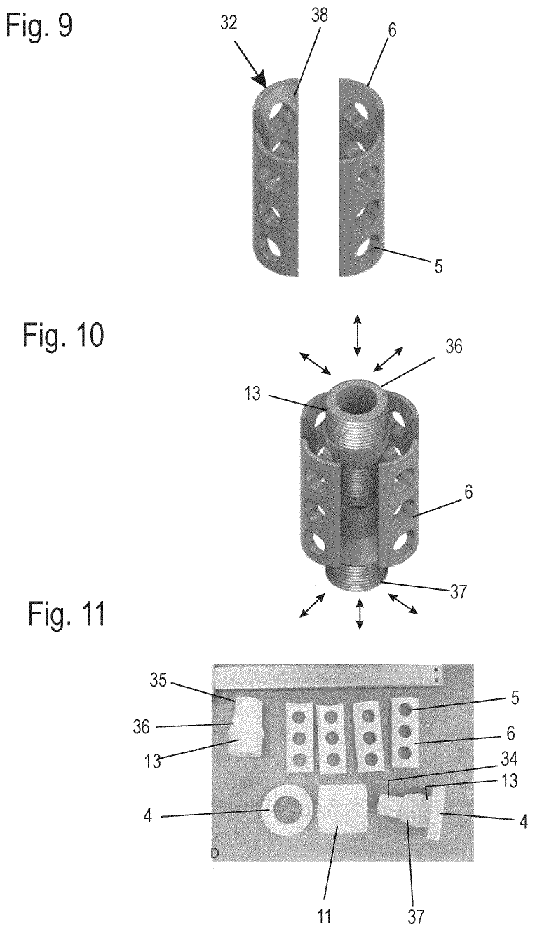

According to FIG. 9 each arcuated plate 6 is provided with a slant 38 at both ends of the plate 6, thus reducing the plate thickness at this position. The slant 38 provides a sliding surface, which is in contact with the conical sections 36, 37.

FIG. 8 illustrates the complete RTCI 40. The scaffold holds the plates 6 from falling apart. Wherein the conical parts perform a stretching force on the plates.

The above-described mechanism of the RTCI for a radial stretching of the scaffold 11 is a further feature of the invention that can realized independent from the provision of the protrusions 4. However, the combination of both features provides a combination of two options for a controlled growth of the tissue on the surface of the scaffold 11 within one insert.

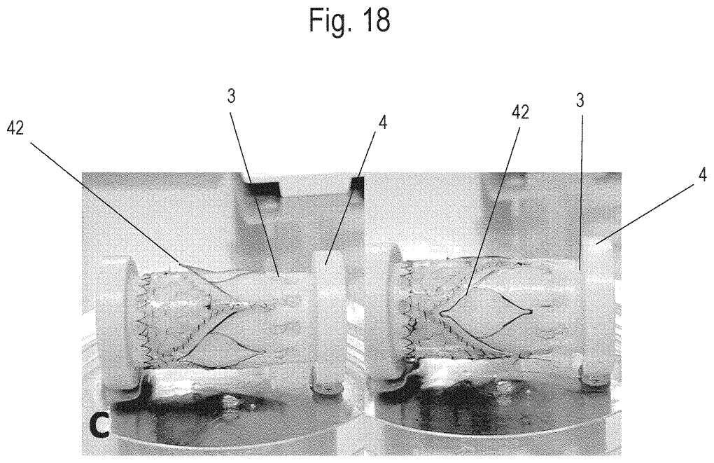

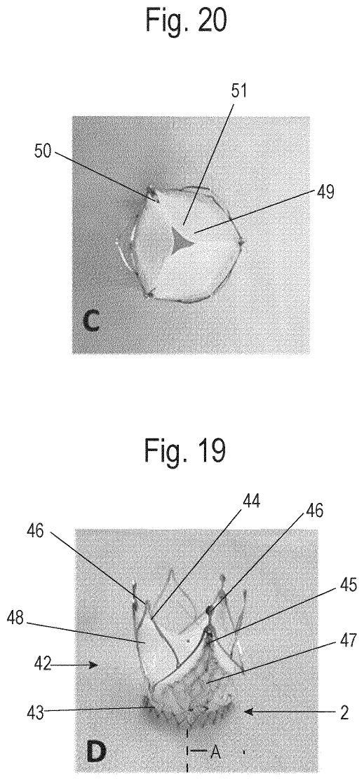

A further object of the invention is the variation of the structure of the substrate 42 compared to the aforementioned first and second embodiment as shown in FIGS. 18 and 19. The substrate in this case is a cylindrical mesh body as described in the first embodiment, however the substrate 2 is provided with meshes of different sizes. The mesh body 42 is provided with a circumferential mesh section 43 defining a central axis A with meshes having approximately uniform size. The mesh body 42 is further provided with prongs 44, 45 and a having a tapered end 46, wherein the prongs 44, 45 extend parallel to the central axis A from the mesh section 43 towards its end. One prong 44 has one large mesh 48 having a mesh size of at least 3 times as big as the average mesh size of the meshes of the mesh section 43. The second prong 45 is provided by a mesh structure 47 with meshes having the same average mesh size as the mesh section 43.

This way the second prong 45 is stiffer than the first prong 44. Once the tissue 51 is formed at the surface or around the substrate 42, the section 49 of the tissue 51 formed at or around the surface of the first prongs 44 are more bendable towards the central axis A than the section 50 of the tissue 51 formed at or around the surface of the second prongs 45. As shown valve flaps are realized by modification of the structure 42 as shown in FIG. 20.

Further a use case, further advantages of the invention and some further embodiments are further described below.

Cardiovascular tissues, such as blood vessels and heart valves, are characterized by highly organized extracellular matrix, also called ECM. Among others, the ECM orientation is crucial in determining the mechanical properties of the tissue and its capability in sustaining the hemodynamic loading. Usually, to achieve a strong tissue engineered (TE) construct compatible with cardiovascular tissue engineering applications, such as TE heart valves, TEHV or vascular grafts, complex bioreactor systems are used to mechanically condition the cells by applying physiological-like flow and/or pressure conditions that lead to preferential collagen orientation.

The invention provides a novel and simpler method to produce TE samples with the ability to control the preferential ECM orientation. To fulfill this aim, a new tissue culture method compatible with commercially available roller bottle systems, such as the commercially available system “CellRoll” of the Pfeiffer company, has been developed to favor homogeneous ECM deposition over tissue culture time by ensuring medium perfusion and oxygenation. This new culture method is performed in combination with three different options designed to control ECM orientation and achieve mechanically robust TEHVs:
A first option is the provision of a RTC insert, also called RTCI, wherein the aforementioned protrusions have the function of two wheels as shown in the first, second, and third embodiment. This system allows a preferential longitudinal ECM alignment in response to the static stretch determined by the way the scaffold is sutured or fixed otherwise onto the scaffold holder. The resulting TEM can be used for TEHV manufacturing.

A second option is the provision of a stretcher in radial direction for example with a screw-like mechanism and sliding components to manually increase its diameter and an expandable stent-like scaffold holder where the scaffold is sutured onto or otherwise fixed. By imposing of a continuous or step-wise increase of the stretcher diameter over tissue culture time, a preferential circumferential ECM orientation can be achieved. The resulting TEM can be used for TEHV manufacturing. The sleeve can be part of the stretcher cooperating with the axle body.

A third option is the provision of the stent-like scaffold holder that used is a nitinol stent specifically designed for minimally invasive transcatheter aortic valve replacement, short TAVR, techniques. This option will allow the tissue engineered matrix (TEM) to grow directly on the TAVR stent that will be used for implantation, thereby significantly simplifying TEHV manufacturing by reducing TEM handling and suturing or other kind of fixation.

The resulting RTCI is then seeded with cells and inserted in a preferably pleated roller-bottle for tissue culture in a commercially available roller bottle system.

As mentioned before some or all parts of the rotating tissue culture insert of the aforementioned embodiments could be made of medical grade materials such as stainless steel. Alternatively, a suitable material can be a 3D-printed polymer material and/or a glass-fiber enriched polymer materials, such as a nylon blend.

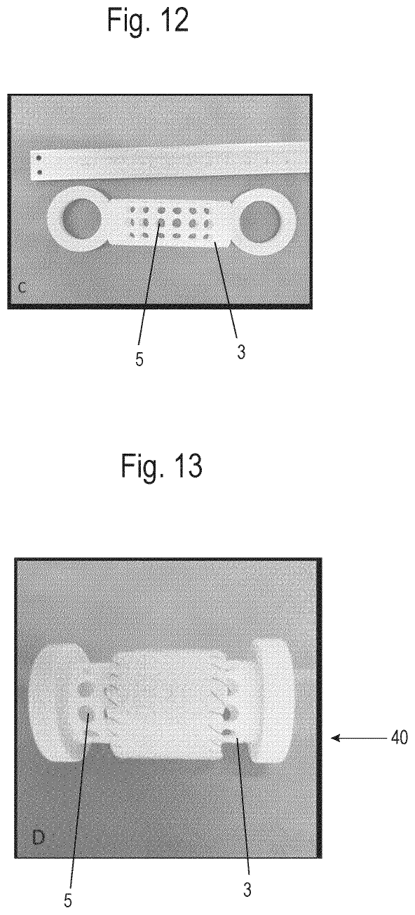

FIGS. 12 and 13 show a fourth embodiment of the invention, wherein the hollow axle body 3 is modified such way that it is provided with more openings than in FIG. 1-6. The free space defined by the openings in this embodiment is at least 30% of the outer cylindrical surface of the axle body 3, whereas the free space defined by the openings 5 of the first embodiment is less than 10% of the outer cylindrical surface of the axle body.

Two prototypes of the RTCI for option 1 according to FIG. 1-6 and FIG. 12-13 and one prototype for the RTCI stretcher according to FIG. 7-11 have been manufactured and tested in multiple cultures.

For all the experiments, a scaffold based on polyglycolic acid (PGA) coated with 1% P4HB has been used. For the testing, a rectangular scaffold (size: 9.5×4 cm²) was sutured onto a 28 mm stent-like scaffold holder and then inserted either into the RTCI described for option 1 (n=5 for each time point of 2, 4, and 6 weeks of tissue culture)—see FIG. 1-6, or into the stretcher described in option 2 (n=4 constructs cultured for 6 weeks)—see FIG. 7-11. Neonatal human dermal fibroblasts (hDFBs, passage 7-9) have been seeded at 1×10⁶/cm² using fibrin as a cell carrier, as per standard protocol.

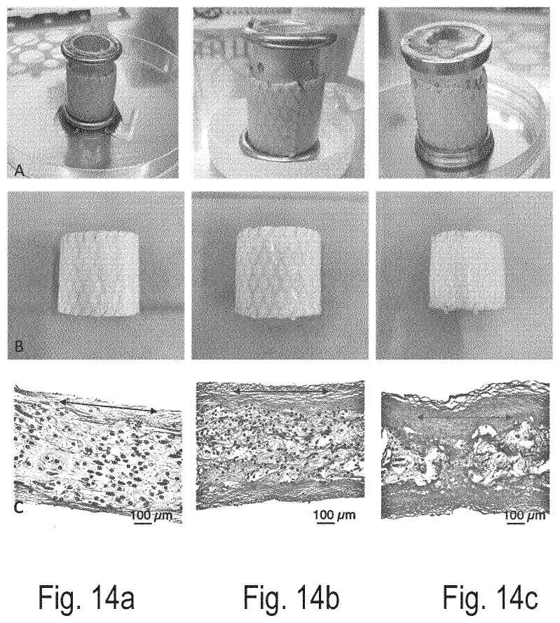

The Results using option 1 are shown in FIG. 14a-14c. TEMs were manufactured using different tissue culture time points to better evaluate the performance of the rotating tissue culture method using the RTCI described in option 1. The following samples have been manufactured and tested by gross evaluation, immunohistology, mass spectroscopy, and uniaxial mechanical testing.

FIG. 14 a discloses n=6 tubes cultured for 2 weeks

FIG. 14 b discloses n=6 tubes cultured for 4 weeks

FIG. 14c discloses n=6 tubes cultured for 6 weeks

FIG. 14 a-c disclose representative histological images of TEM longitudinal cross sections indicating an increase in ECM deposition and organization over tissue culture time. Importantly, ECM is prevalently aligned in the longitudinal direction, as indicated by the blue arrow.

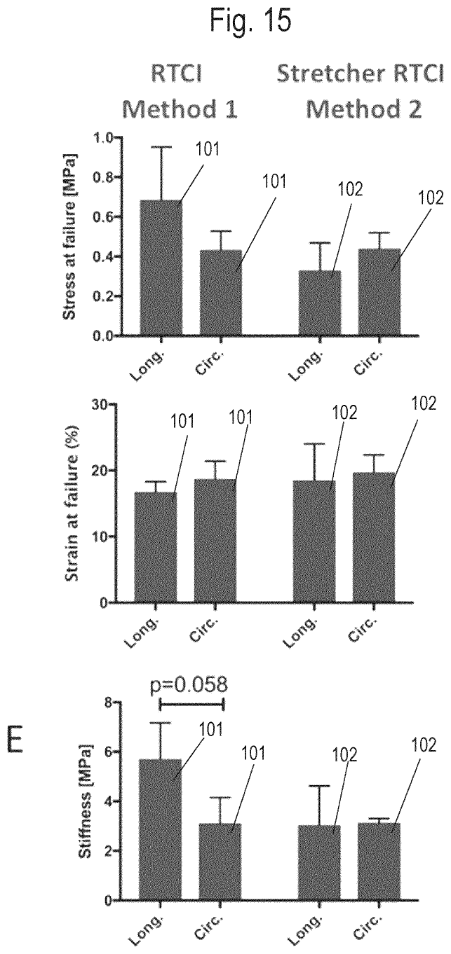

Further the results using option 2 and option 1 were compared. To compare the performance of the RTCI of FIG. 1-6 and the stretcher RTCI of FIG. 7-11, TEMs were manufactured using 6 weeks tissue culture time. N=4 TEM samples have been manufactured using the stretcher RTCI, and tested by gross evaluation, immunohistology, and uniaxial mechanical testing and compared to the results of FIG. 14a-c. Results were compared to the TEMs cultured using the RTCI described in option 1. FIG. 15 shows results after 6 weeks of culture, wherein the resulting TEM is decellularized and removed from the RTCI and scaffold holder for further testing.

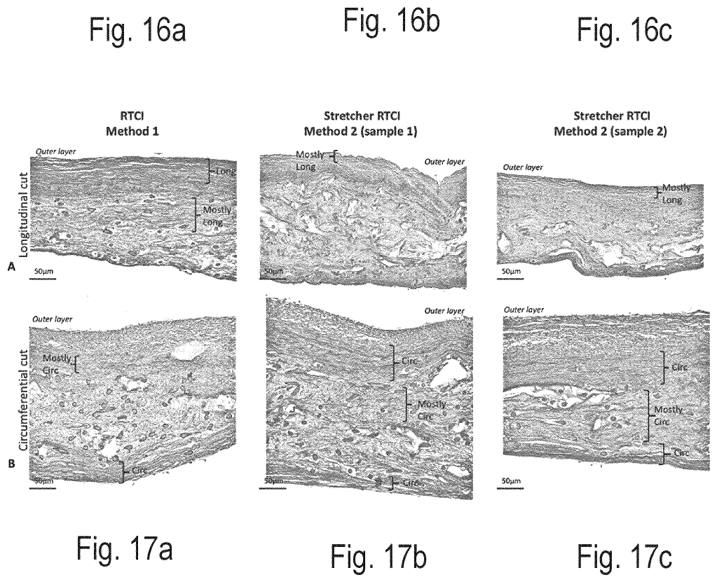

FIG. 15 disclose the results of the uniaxial tensile test performed on TEM samples manufactured using the stretcher RTCI of FIG. 7-11 having the ref. sign 101 or the RTCI described in option 1 of FIG. 1-6 having the ref. sign 102. Uniaxial mechanical testing of both circumferential and longitudinal samples provided information on tissue anisotropy. Stress at failure and stiffness are greater in the longitudinal direction for TEM tubes cultured using option 1. On the other hand, stress at failure and stiffness are slightly greater in circumferential direction for TEM tubes cultured using the stretcher described in option 2. These results indicate a shift in mechanical properties that can be associated to different ECM orientation as shown in FIG. 16a-c and 17a-c.

FIG. 16 a-c disclose a histological evaluation of samples cultured for 6 weeks using the RTCI of option 1, also called method 1, and the stretcher RTCI of option 2, also called method 2. The FIG. 16 a-c are histological images of samples cut in the longitudinal direction.

FIG. 17 a-c disclose histological images of samples cut in the circumferential direction. This analysis indicates that option 1 results in mostly longitudinal ECM alignment. On the other hand, the stretcher of option 2 or method 2 results in mostly circumferential collagen alignment, in particular within the central portion of the TEM.

Option 3 together with FIG. 18-20 discloses the potential and relevance of the current invention for cardiovascular tissue engineering. FIG. 18-20 is a result after 6 weeks of culture using the a RTCI according to option 1, the resulting TEM was used to manufacture tissue engineered heart valves with TEHVs, n=6, mounted on a nitinol stent compatible with transcatheter aortic valve replacement TAVR-techniques. The valve was then inserted into a custom-made silicon holder with inner diameter of 25 mm, and then tested in-vitro in a pulse duplicator system at both pulmonary—(peak systolic pressure: 20 mmHg) and aortic-like pressure conditions (peak systolic pressure: 120 mmHg) for a minimum of 1 hour with good results. Similarly, after 6 weeks of culture using the RTCI of option 2 the resulting TEM could also be used to manufacture TEHVs, n=2. In vitro testing in the pulse duplicator system indicated good performance at pulmonary pressure conditions for 1 hour.

Next, a detailed way for the preparation of a tissue by using the RTCI is described below:

### Scaffold Preparation

hTEMs were produced as previously described by using non-woven polyglycolic acid (PGA) meshes (thickness 1 mm; specific gravity 70 mg/cm3 ; Confluent) coated with 1% poly-4-hydroxybutyrate (P4HB, MW 1×10⁶; TEPHA Inc.) in tetrahydrofuran (Sigma-Aldrich) as starting matrix. The PGA/P4HB mesh was cut into scaffolds (width: 100 mm; height: 45 mm) and then sutured on a cylindrical scaffold holder (30mm in height, 28 mm inner diameter) based on a nitinol stent. Afterwards, the scaffold is combined to the RTC insert according to FIG. 1-6 and the full constructs is sterilized (30 min incubation in 70% ethanol followed by 30 min incubation in PBS supplemented with 10% penicillin-streptomycin (Sigma) and 1% antibiotic-antimicotic solution (Sigma). Finally, the scaffolds were incubated overnight in cell culture medium (Advanced DMEM (Gibco), supplemented with 10% fetal bovine serum (FBS, Gibco), 1% GlutaMax (Gibco), and 1% penicillin-streptomycin (Sigma).

### Cell Expansion and Seeding

Human dermal fibroblasts (hDFBs, CellSystems Biotechnologie GmbH, passages 7-10) were plated in roller bottles (850 cm², TufRol EZ, Nunc) and expanded in cell culture medium until confluency using the CELLROLL system. Upon harvesting, cells were seeded onto the scaffolds (1×10⁶ cells/cm²) using fibrin as a cell carrier, as previously established. After seeding, the constructs were incubated statically in cell culture medium overnight to favour cell adhesion.
hTEM Culture
The day after the seeding, the constructs were transferred to a pleated roller-bottle (1450 cm² , TufRol, Nunc). Constructs were cultured using 150 ml of tissue culture medium (cell culture medium supplemented with I-ascorbic acid 2-phosphate (0.26 mg/ml; Sigma-Aldrich) from day 1, and 5 ng/ml TGF-ß1 (Peprotech), starting at day 7), that was replaced twice a week. Tissue culture occurred using a rotation speed of 1.5 rpm in standard incubator settings (37° C., 5% CO₂, and 95% relative humidity). After 2, 4, or 6 weeks of tissue culture, hTEM tubes were washed in PBS and decellularized using an optimized protocol.

### Decellularization

hTEM were incubated twice overnight in a detergent solution (0.25% Triton X-100, sodium deoxycholate and 0.02% ethylenediaminetetraacetic acid in DPBS) in the roller bottle. Additionally, four Benzonase (Novagen) incubation steps with decreasing concentration (100, 80, 40 and 20 U/ml in 50 mM TRIS-HCl buffer solution with pH 8.0) were used to degrade the remaining DNA remnants. After rinsing, hTEM tubes were stored in PBS supplemented with 1% penicillin/streptomycin (Sigma) at 4° C. until further use.

### (Immuno-)histochemistry

Qualitative, quantitative, and (semi)quantitative immune-histological evaluation of the cross-sections of the different human-cell derived TEMs (hTEMs) manufactured was used to gain relevant information on ECM structure and deposition, as well as on the presence of scaffold remnants.
For each tube, a longitudinal portion of the hTEM was cut and fixed in 4% formalin, dehydrated, embedded in paraffin, and cut longitudinally (slice thickness of 3 μm). The resulting cross-section was stained using: hematoxylin and eosin (H&E), to evaluate the presence of cells and the tissue morphology; Elastica van Gieson (ELVG) to examine the elastic and collagen fibers; and collagen 1 (COL1, Abcam, ab34710) and collagen 3 (COL3, Abcam, ab7778) to assess the deposition of specific collagens. The stained samples were imaged with the brightfield microscopy (Mirax Midi Microscope, Carl Zeiss GmbH).
Assessment of hTEM Dimensions
Total hTEM thickness and ECM thickness on the outer and inner layer of the scaffold were measured using the Pannoramic Viewer software (3DHistech, Ltd.). For each hTEM sample, thickness measures were obtained from ten different locations per section and then averaged. For each tissue culture time point (i.e.: 2, 4, and 6 weeks), n=4 different hTEM samples have been measured and the averaged value reported.

### Semi-Quantitative Evaluation

A semi-quantitative evaluation of the histology was performed on each sample by grading from 0 (absent) to 5 (abundant) the maturation and presence of ECM (in the outer, inner, and middle region of the hTEM sample), the presence of polymeric remnants, the efficiency of the decellularization, and the presence of collagen 1, 3, and elastin. The analysis was performed by two independent observers for each hTEM sample. For each tissue culture time point (i.e.: 2, 4, and 6 weeks), n=4 different hTEM samples have been analysed.

### Biochemical Analysis

Biochemical assays were used to quantify collagen (via hydroxyproline, HYP) and glycosaminoglycans (GAGs) content. Briefly, 5-8 mg of freeze-dried hTEM samples (n=4 for each time point, in technical triplicate) were digested with papain (Worthington) in a digestion buffer solution and a stepwise protocol was performed to quantify GAGs and HYP content using a plate reader (Tecan infinite M1000 pro) to measure the light absorbance. Quantification was performed by using standards for GAGs and HYP. Double strand DNA (dsDNA) analysis was performed using the Qubit dsDNA HS Assay kit (Thermofisher). The data was normalized to total weight of dry tissue.
Uniaxial Tensile Testing The uniaxial mechanical testing was performed to gain information on how mechanical properties changes over tissue culture time. hTEM samples in longitudinal direction (n=3 for each tissue culture time point, in technical triplicate) were cut at a length of 9×5 mm, clamped into a custom-made clamp before being tested using a uniaxial material testing machine that recorded force-strain data (Zwick Z010 TN, ZwickRoell GmbH, Ulm, Germany-20N load-cell). During testing, samples were kept in a chamber filled with room temperature PBS. After an initial preload to 0.025 N (0% strain), samples were pre-conditioned five times to 5% strain, with a recovery period of 30 s in between each preloading cycle. Then, samples were ramped to failure at a constant strain rate of 100% strain per min. Stress calculation was achieved by using an estimate of the sample cross-sectional area calculated as follow: sample width (5 mm) multiplied by tissue thickness. Tissue thickness was obtained on the basis of histological images of adjacent samples. The thickness value was then corrected by a volume shrinkage correction factor of 1.15 to compensate for loss of tissue volume due histological sample preparation. Sample stiffness (E, Young's modulus), stress at failure and strain at failure were calculated in the linear region of the force-strain curves.

### Heart Valve Manufacturing and In Vitro Testing

Systematic in-vitro assessment of TEHV performance (n=4) was carried out by using a pulse duplicator system HDT-500 (BDC Labs, USA) equipped with a transonic sensor TS410 (Transonic Systems, USA) for flow measurements and a BDC-PT pressure sensor (BDC Labs, USA). All TEHVs were firstly tested at simulated pulmonary pressure conditions (peak pressure: 20 mmHg, mean pressure: 10 mmHg, minimum pressure: 0 mmHg) for 1 hour. After a gradual increase of the applied pressure, the TEHVs were tested at simulated aortic pressure conditions (peak pressure: 120 mmHg, mean pressure: 100 mmHg, minimum pressure: 80 mmHg) for 1 hour. Testing was performed in PBS supplemented with 0.05% Xanthan Gum (Sigma), in accordance with ISO 5840 testing requirement. Data were collected for 3 seconds and functionality was assessed by averaging 20 simulated cardiac cycles using the Statys software (BDC Labs, USA) to determine the closing volume (CV, back flow during valve closure), leakage volume (LV,) and regurgitation fraction (RF) expressed as % of the Forward Flow Volume (FFV); the Effective Orifice Area (EOA, estimated using the Gorlin equation, as described in the ISO5840); and maximum and mean transvalvular pulmonary/aortic gradient (peak-pressure difference, PPDₘₐₓ and PPDₘₑₐₙ, respectively, calculated as the difference between ventricular pressure and pulmonary/aortic pressure at systole).

### LIST OF REFERENCES

2 scaffold holder
3 axle body
4 protrusions
5 openings
6 plates
7 axle body part
8 axle body part
11 scaffold
12 distal ends
13 threads
20 rotating tissue culture insert
25 surface
27 ribbons
32 sleeve
33 axle body
34 screw bolt
35 screw sleeve
36 conical section
37 conical section
38 slant
40 RTCI
42 mesh body
43 mesh section
44 prong
45 prong
46 tapered end
47 mesh structure
48 large mesh
49 section
50 section
51 tissue
100 bottle
101 sign
102 sign

1-15. (canceled)

16. A rotating tissue culture insert comprising:
a scaffold configured to cultivate cells;
an axle body; and
at least two protrusions extending in a radial direction from the axle body,
wherein the rotating tissue culture insert is configured to be positioned inside a rotatable roller bottle partially filled with a liquid to cultivate the cells, and
wherein the at least two protrusions are wheels of the rotating tissue culture insert with a runway that is in contact with a surface of a roller bottle when the roller bottle is in a lying position.

17. The rotating tissue culture insert of claim 16, wherein the at least two protrusions are at a distal position of the axle body.

18. The rotating tissue culture insert of claim 16, wherein a radial height of the at least two protrusions is larger than
a thickness of the scaffold, or
a sum of the thickness of the scaffold and a scaffold holder.

19. The rotating tissue culture insert of claim 18, further comprising:
a sleeve coaxial to and positioned over the of the axle body,
wherein a longitudinal axis of the sleeve is parallel to the axle body,
wherein the sleeve comprises a plurality of openings, and
wherein
the scaffold holder is positioned over the sleeve or the axle body and is a part of the rotating tissue culture insert, or
the axle body or the sleeve is a hollow body comprising a plurality of openings.

20. The rotating tissue culture insert of claim 16, wherein the at least two protrusions are demountable from the axle body.

21. The rotating tissue culture insert of claim 16, wherein the at least two protrusions a form of circumferential rings.

22. The rotating tissue culture insert of claim 16, wherein the scaffold comprises a nonwoven layer.

23. The rotating tissue culture insert of claim 16, further comprising:
a stretcher configured to stretch the scaffold in radial direction.

24. The rotating tissue culture insert of claim 19, wherein the axle body comprises a longitudinal axis and wherein the rotating tissue culture insert further comprises one or more parts forming the sleeve and that are radially movable in relation to the longitudinal axis.

25. The rotating tissue culture insert of claim 24, further comprising:
an adjustment mechanism configured to adjust a radial distance of the one or more parts relative to the longitudinal axis of the axle body.

26. The rotating tissue culture insert of claim 25, wherein the axle body is part of the adjustment mechanism and is formed by at least two elements that are linear movable to relative each other.

27. The rotating tissue culture insert of claim 25, wherein the adjustment mechanism is adjustable by screwing.

28. The rotating tissue culture insert of claim 18, wherein the scaffold holder is cylindrical and comprises different sections of bendability.

29. A method for producing of an implant comprising, the method comprising:
a. providing a roller bottle comprising a liquid for cultivating cells and a rotating tissue culture insert, wherein the rotating tissue culture insert comprises a scaffold configured to cultivate the cells, an axle body, and at least two protrusions extending in a radial direction from the axle body, wherein the rotating tissue culture insert is positioned inside the roller bottle, wherein the at least two protrusions are wheels of the rotating tissue culture insert with a runway that is in contact with a surface of a roller bottle when the roller bottle is in a lying position such that a fill level of the roller bottle is parallel to a longitudinal axis of the bottle, wherein the rotating tissue culture insert further comprises a scaffold holder attaching the scaffold to the rotating tissue culture insert;
b. rotating the roller bottle, wherein the scaffold is distanced towards a wall of the roller bottle, wherein the rotating tissue culture insert rotates in the roller bottle, and wherein a tissue is formed at a surface of the scaffold during the rotation of the roller bottle; and
c. removing an arrangement comprising at least the tissue and the scaffold holder from the axle body of the rotating tissue culture insert, wherein the arrangement is an integral part of the implant.

30. The method of claim 29, wherein the arrangement comprises a cylindrical section, wherein a radial diameter of the rotating tissue culture insert is adjustable, and wherein the radial diameter of the rotating tissue culture insert is adjusted to a variable diameter of the cylindrical section.
